(19) United States
(12) Patent Application Publication
(10) Pub. No.: US 2022/0354434 A1
(43) Pub. Date: Nov. 10, 2022

(54) BIOLOGICAL INFORMATION MEASURING DEVICE

(71) Applicant: Sumitomo Riko Company Limited, Aichi (JP)

(72) Inventors: Masaru Murayama, Aichi (JP); Hirokazu Yamamoto, Aichi (JP); Naoya OUE, Aichi (JP); Ryo Shimura, Aichi (JP)

(73) Assignee: Sumitomo Riko Company Limited, Aichi (JP)

(21) Appl. No.: 17/868,755

(22) Filed: Jul. 19, 2022

Related U.S. Application Data
(63) Continuation of application No. PCT/JP2021/032826, filed on Sep. 7, 2021.

(30) Foreign Application Priority Data
Sep. 30, 2020 (JP) 2020-164428

(51) Int. Cl. A61B 5/00

(52) U.S. Cl. CPC A61B 5/7221; A61B 5/7239; A61B 5/7246; A61B 5/6893; A61B 5/7225; A61B 5/726; A61B 5/024

(57) ABSTRACT

A biological information measuring device comprises: a plurality of sensors that each acquire a base signal containing biological information and noise information; and a processing device that acquires biological information on the basis of a plurality of base signals. The processing device comprises: a component analysis part that performs a prescribed component analysis on the basis of the plurality of base signals and generates a plurality of component signals which constitute the plurality of base signals; and a biological information acquisition part that determines whether a component signal is biological information.

### CROSS-REFERENCE TO RELATED APPLICATION

The present application is a continuation of PCT/JP2021/032826, filed on Sep. 7, 2021, and is related to and claims priority from Japanese patent application no. 2020-164428, filed on Sep. 30, 2020. The entire contents of the aforementioned application are hereby incorporated by reference herein.

### TECHNICAL FIELD

The disclosure relates to a biological information measuring device.

### RELATED ART

Patent Literature 1 describes a measuring device that simultaneously detects a body pressure distribution and a pulse wave of a subject. Patent Literature 2 describes that biological information such as heart rate and respiratory rate is calculated based on a value detected by a pressure sensor cell generated by a subject.

Patent Literature 3 describes that the average of each light wavelength component is calculated from the image data captured by the subject based on the time series data of multiple light wavelength components, and an independent component analysis is applied to the average to obtain multiple independent signals, and the heart rate and the respiratory rate are detected from the obtained multiple independent signals.

Patent Literature 4 describes a blood pressure measuring device including multiple identification parts which, based on the relationship between blood pressure and the feature amount of biological information obtained by pre-training for each predetermined blood pressure, binarizes and determines with respect to the feature amount of biological information whether the blood pressure corresponding to the feature amount is less than or greater than or equal to a predetermined blood pressure; and a binarization determination part which, when estimating the blood pressure, binarizes and determines multiple different predetermined blood pressures with respect to the feature amount of the biological information obtained by the measurement using the identification parts.

Patent Literature 5 describes a principal component analysis is performed on time-series data of detection signals from multiple pressure sensors to calculate a mode vector corresponding to a reception gain of a respiratory signal. Patent Literature 6 describes that analysis processing such as an independent component analysis, a principal component analysis, and a singular value decomposition is performed on multiple pieces of extracted data extracted under multiple extraction conditions.

Patent Literature 7 describes that a neural network is used in which a measured pulse wave signal is input and trained to reproduce a pulse wave having an amplitude peak synchronized with the heartbeat of a living body, and a pulse rate is calculated from the pulse wave reproduced by the neural network. Patent Literature 8 describes that the biological information of a subject is acquired by inputting into a pre-trained trained model for acquiring the biological information representing the state of the subject from the measurement information.

### CITATION LIST

### Patent Literature

[Patent Literature 1] Japanese Patent Application Laid-open No. 2017-176498.
[Patent Literature 2] Japanese Patent Application Laid-open No. 2017-176499.
[Patent Literature 3] Japanese Laid-open Patent Publication No. 5672144.
[Patent Literature 4] Japanese Laid-open Patent Publication No. 5218139.
[Patent Literature 5] Japanese Patent Application Laid-open No. 2017-140187.
[Patent Literature 6] International Patent Application Laid-open No. 2019-208388.
[Patent Literature 7] Japanese Laid-open Patent Publication No. 4320925.
[Patent Literature 8] Japanese Patent Application Laid-open No. 2020-48674.

### SUMMARY

### Technical Problem

By the way, when measuring the biological information of the occupants of a vehicle, since the vibrations of the vehicle itself are detected as noise, it is not easy to measure the biological information with high accuracy. In particular, when the vehicle is traveling, the frequency band of the vibrations accompanying the traveling is partly common to the frequency band of human biological information; therefore, biological information and noise information cannot be separated by frequency filters (such as a bandpass filter).

The disclosure has been made in view of such issues, and provides a biological information measuring device capable of measuring biological information with high accuracy by performing a process capable of distinguishing biological information and noise information.

### Solution to Problem

In an embodiment of the disclosure, provided is a biological information measuring device, including:

multiple sensors that acquire base signals including biological information and noise information; and

a processing device that acquires the biological information based on the base signals, and the processing device includes:
a component analysis part that performs a predetermined component analysis based on the base signals to generate multiple component signals configuring the base signals; and
a biological information acquisition part that determines whether the component signal is the biological information.

### Effects of Invention

The component analysis part of the processing device performs a predetermined component analysis based on the multiple base signals to generate multiple component signals configuring the base signals. That is, a part of the generated multiple component signals becomes signal mainly configured by biological information, and other parts become signals mainly configured by noise information. That is, even if the base signal includes noise information in addition to the biological information, the component signals are signals in which the biological information and the noise information are separated.

Then, the biological information acquisition part of the processing device determines whether the component signal is the biological information. That is, the biological information acquisition part determines which component signal among the multiple component signals is a signal mainly configured by biological information by making a determination for each of the component signals. Therefore, the biological information measuring device may measure the biological information with high accuracy.

### BRIEF DESCRIPTION OF THE DRAWINGS

FIG. 1 is an overall configuration view of a biological information measuring device.

FIG. 2 is an illustration view of a mounting position of a sensor unit.

FIG. 3 is an exploded perspective view of the sensor unit.

FIG. 4 is a graph showing base signals A.

FIG. 5 is a functional block configuration diagram of a biological information measuring device.

FIG. 6 is a functional block configuration diagram of a pre-processing part configuring a biological information measuring device.

FIG. 7 is a functional block configuration diagram of a post-processing part configuring a biological information measuring device.

FIG. 8 is a graph showing component signals C.

FIG. 9 is a graph showing power spectra D of the component signals C.

FIG. 10 is a diagram showing candidates of feature amounts.

FIG. 11 is a diagram showing candidates of feature amounts.

FIG. 12 is a diagram showing candidates of feature amounts.

FIG. 13 is a diagram showing candidates of feature amounts.

FIG. 14 is a flowchart showing a process of a biological information acquisition part configuring a biological information measuring device.

FIG. 15 is a diagram showing a softmax function.

FIG. 16 is a graph in which secondary candidates at each time are plotted in a predetermined time range.

FIG. 17 is an enlarged graph in the range of time 200 msec to 300 msec in the graph of FIG. 16.

FIG. 18 is a graph showing a continuous line connecting each plot point in a predetermined time range.

FIG. 19 is an enlarged graph in the range of time 200 msec to 300 msec in the graph of FIG. 18.

FIG. 20 is a graph showing a continuous line after filtering in a predetermined time range.

FIG. 21 is an enlarged graph in the range of time 200 msec to 300 msec in the graph of FIG. 20.

### DESCRIPTION OF THE EMBODIMENTS

(1. Configuration of Biological Information Measuring Device 1)

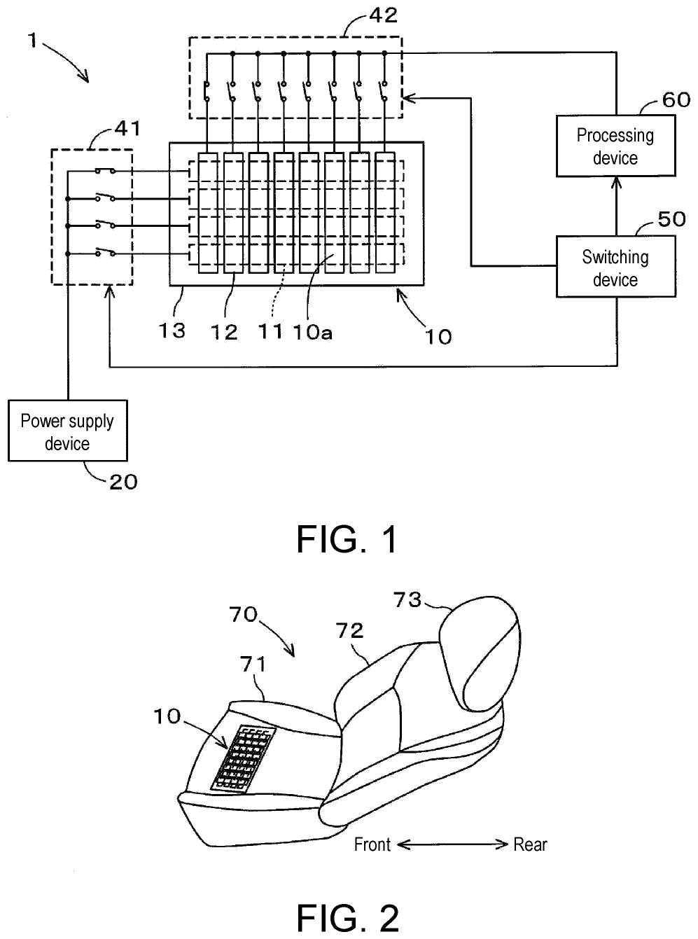

The configuration of the biological information measuring device 1 (hereinafter referred to as a measuring device) will be described with reference to FIGS. 1 to 3. The measuring device 1 measures the biological information of an occupant seated on a seat of a vehicle regardless of whether the vehicle is traveling or not. In particular, the measuring device 1 is useful in that it can measure biological information while the vehicle is traveling. Here, while the vehicle is traveling, vibrations accompanying the traveling occur. That is, the measuring device 1 can measure the biological information of the occupant even when it is subjected to the vibrations generated by the traveling of the vehicle. Further, certainly, the measuring device 1 can measure biological information while the vehicle is stopped.

The measuring device 1 measures the biological information of the body given to a sensor unit 10 formed in a planar shape (equivalent to a sheet shape or a film shape). The measuring device 1 measures at least one of a heart rate and a respiratory rate as biological information. As shown in FIG. 1, the measuring device 1 includes a sensor unit 10, a power supply device 20, switch circuits 41 and 42, a switching control device 50, and a processing device 60.

In this example, the case where the sensor unit 10 is configured by multiple capacitance sensors will be described as an example. The sensor unit 10 may use other sensors such as a piezoelectric sensor and a Doppler sensor. In this case, a measuring device may be configured according to each type of sensor.

As shown in FIG. 2, the sensor unit 10 is disposed inside, for example, on a front side of a seat surface 71 of a seat 70. Specifically, the sensor unit 10 is disposed on the back surface side of the coverings on the front side of the seat surface 71. That is, the sensor unit 10 is influenced by the pulse wave of the femoral arteries of the occupant, the respiratory component, and the like.

In addition to being disposed on the front side of the seat surface 71 of the seat 70, the sensor unit 10 may be disposed on the rear side the seat surface 71, on a back surface 72, or on a headrest 73. When the sensor unit 10 is disposed on the rear side the seat surface 71, the sensor unit 10 receives body pressure from the occupant's buttocks and is influenced by the pulse wave of the arteries in the occupant's buttocks, the respiratory component, and the like. Further, when the sensor unit 10 is disposed on the back surface 72, the sensor unit 10 receives body pressure from the occupant's back and is influenced by the pulse wave of the arteries in the occupant's back, the respiratory component, and the like. Further, when the sensor unit 10 is disposed on the headrest 73, the sensor unit 10 receives body pressure from the head of the occupant and is influenced by, for example, the pulse wave of the arteries in the neck, the respiratory component, and the like.

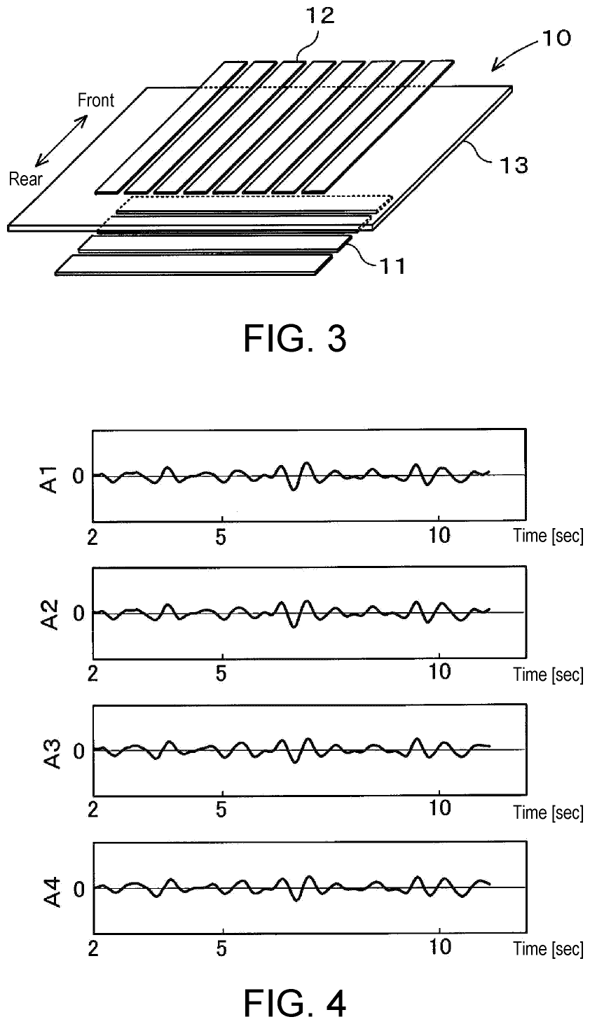

The detailed configuration of the sensor unit 10 will be described with reference to FIGS. 1 and 3. The sensor unit 10 has, for example, flexibility and is formed in a planar shape (equivalent to a sheet shape or a film shape). The sensor unit 10 may be compressed and deformed in the plane normal direction. For example, the sensor unit 10 includes four rows of first electrodes 11, eight rows of second electrodes 12, and a dielectric layer 13. The number of rows of the first electrodes 11 and second electrodes 12 may be changed as appropriate. The dielectric layer 13 is formed in an elastically deformable planar shape, and is sandwiched and disposed between the first electrode 11 and the multiple second electrodes 12.

Each first electrode 11 is formed in a band shape and is disposed parallel to each other. The extending direction of the first electrode 11 coincides with the left-right direction of the seat 70 in FIG. 2. The second electrode 12 is disposed in the plane normal direction of the sensor unit 10 at a distance from the first electrode 11. Each second electrode 12 is formed in a band shape and is disposed parallel to each other. The extending direction of the second electrode 12 coincides with the front-rear direction of the seat 70 in FIG. 2. That is, on the seat surface 71 of the seat 70, the second electrodes 12 are disposed in four rows on each of the left and right sides. The second electrodes 12 in the left four rows are located at a position corresponding to the left thigh of the occupant, and the second electrodes 12 in the right four rows are located at a position corresponding to the right thigh of the occupant. Then, the extending direction of each second electrode 12 coincides with the extending direction of the thigh part, and thus coincides with the extending direction of the femoral arteries.

The first electrode 11 and the second electrode 12 are formed by filling a conductive filler in an elastomer. The first electrode 11 and the second electrode 12 have flexibility and stretchability. The dielectric layer 13 is formed by an elastomer and has flexibility and stretchability.

Therefore, the facing positions of the first electrode 11 and the second electrode 12 are located in a matrix. In this example, the matrix-like facing positions include 32 (=4×8) points. The sensor unit 10 includes a pressure sensor cell 10a that functions as a capacitance sensor at multiple (32 points) facing positions disposed in a matrix. As described above, the sensor unit 10 includes 32 pressure sensor cells 10a disposed in 4 rows vertically and 8 rows horizontally. Then, the 32 pressure sensor cells 10a are disposed in a planar shape.

In this example, the pressure sensor cells 10a in the left four rows receive pressure from the left thigh of the occupant, and the pressure sensor cells 10a in the right four rows receive pressure from the right thigh of the occupant. The number of rows of the first electrodes 11 and second electrodes 12 may be freely changed.

Then, when the sensor unit 10 receives a force for compressing in the plane normal direction, the dielectric layer 13 is compressed and deformed, whereby the separation distance between the first electrodes 11 and the second electrodes 12 becomes shorter. That is, the capacitance between the first electrodes 11 and the second electrodes 12 becomes large.

The power supply device 20 generates a predetermined voltage and applies the predetermined voltage to the first electrodes 11 of the sensor unit 10. The switch circuit 41 is configured by multiple switches. One end of each switch in the switch circuit 41 is connected to the power supply device 20, and the other end of each switch is connected to the corresponding first electrode 11. In FIG. 1, the switch corresponding to the first electrode 11 in the first row from the upper side is turned on, and the other switches are turned off.

The switch circuit 42 is configured by multiple switches. One end of each switch of the switch circuit 42 is connected to the corresponding second electrode 12, and the other end of each switch is connected to the processing device 60 (to be described later). In FIG. 1, the switch corresponding to the second electrode 12 in the first row from the left side is turned on, and the other switches are turned off. The switching control device 50 executes ON/OFF switching of each switch of the switch circuits 41 and 42. Then, the switching control device 50 connects the pressure sensor cells 10a which are measurement targets to the power supply device 20 and the processing device 60.

The processing device 60 acquires the heart rate and the respiratory rate, which are biological information, by performing an arithmetic process based on the detection values by the pressure sensor cells 10a which are the measurement targets. Specifically, the processing device 60 calculates the heart rate and the respiratory rate based on the change in the capacitance of the pressure sensor cells 10a.

(2. Processing Configuration of Sensor Unit 10)

As described above, the sensor unit 10 includes 32 (=4×8) pressure sensor cells 10a in a matrix shape. Each of the 32 pressure sensor cells 10a functions as a sensor for measuring capacitance. Therefore, in the following, each of the 32 pressure sensor cells 10a will be referred to as sensors S1 to S32. That is, the sensor unit 10 includes 32 channels (ch) of sensors S1 to S32.

Here, each of the sensors S1 to S32 detects base signals A1 to A32 including biological information and noise information. The amplitude of the biological information is very small. On the other hand, the noise information includes vibrations accompanying the traveling of the vehicle. Therefore, the amplitude of the biological information is smaller than the amplitude of the noise information. Therefore, the base signals A1 to A32 include the biological information having a relatively small amplitude and the noise information having a relatively large amplitude.

Further, each of the base signals A1 to A32 is a signal representing a change in capacitance for a predetermined sampling time length. That is, each of the base signals A1 to A32 has data for a predetermined sampling time length with respect to the magnitude of the change in capacitance at time t. FIG. 4 shows a part of the base signals A1 to A4. The base signals A1 to A32 are waveform data for a predetermined sampling time length.

(3. Configuration of Measuring Device 1)

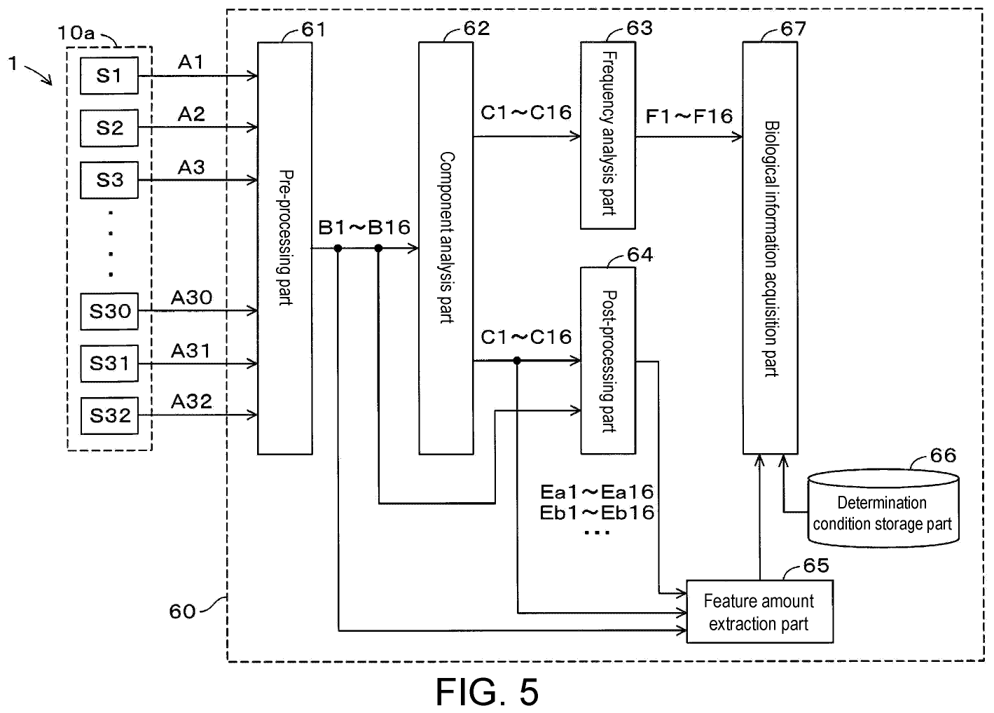

The configuration of the measuring device 1 will be described with reference to FIGS. 5 to 13. However, the measuring device 1 in FIG. 5 shows a functional block configuration diagram for a configuring part including the sensors S1 to S32 and the processing device 60. As shown in FIG. 5, the sensors S1 to S32 acquire the base signals A1 to A32 including the biological information and the noise information.

The processing device 60 acquires the biological information by performing an arithmetic process described below based on multiple (32 channels of) base signals A1 to A32. The processing device 60 includes a pre-processing part 61, a component analysis part 62, a frequency analysis part 63, a post-processing part 64, a feature amount extraction part 65, a determination condition storage part 66, and a biological information acquisition part 67.

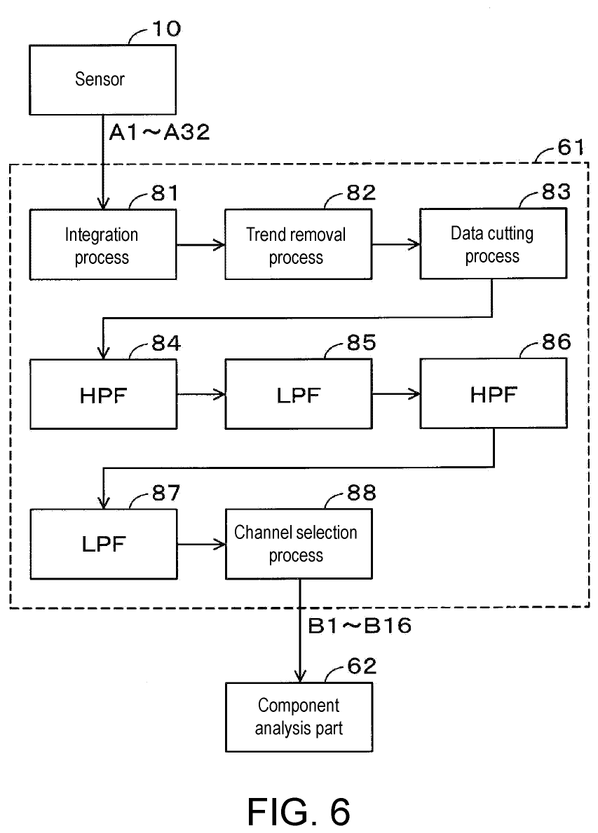

The pre-processing part 61 will be described with reference to FIGS. 5 and 6. As shown in FIG. 5, the pre-processing part 61 acquires multiple (32 channels of) base signals A1 to A32 as input signals. The pre-processing part 61 performs a predetermined pre-process on the multiple base signals A1 to A32 as a pre-process for a predetermined component analysis by the component analysis part 62, and generates multiple (16 channels of) pre-processed signals B1 to B16.

In this example, as shown in FIG. 6, the pre-processing part 61 executes, as a predetermined pre-process, an integration process 81, a trend removal process 82, a data cutting process 83, a first high-pass filter 84, and a first low-pass filter 85, a second high-pass filter 86, a second low-pass filter 87, and a channel selection process 88 (partial signal selection process) are executed.

In this example, the pre-processing part 61 generates multiple (16 channels of) pre-processed signals B1 to B16 by executing all of the above processes 81 to 88. However, the pre-processing part 61 may execute only a part of the above processes 81 to 88, or may execute the processes in a different order. Further, in addition to the above, the pre-processing part 61 may perform a phase difference adjustment process as a predetermined pre-process. The phase difference adjustment process is a process of adjusting multiple signals having different phases so that they may be treated as the same type of signal.

The pre-processing part 61 reduces the noise information as much as possible from the multiple base signals A1 to A32. Further, the pre-processing part 61 selects signals of a part of channels greatly influenced by the biological information from the multiple (32 channels of) base signals A1 to A32. In this example, the pre-processing part 61 selects half (16 channels) of the signals and generates 16 channels of pre-processed signals B1 to B16.

Hereinafter, each of the processes 81 to 88 of the pre-processing part 61 will be described. Here, the base signals A1 to A32 acquired by the sensors S1 to S32 are measured at a predetermined sampling cycle. Therefore, the time required to measure all of the base signals A1 to A32 for 32 channels once is 32 times that time.

The integration process 81 batch-integrates predetermined multiple times for each of the base signals A1 to A32. For example, for the base signal A1, 16 consecutive base signals A1 are added.

The trend removal process 82 is a process for removing a changing DC component. For example, the base signals A1 to A32 of the sensors S1 to S32 may change due to the influence of the change in the posture of the occupant. Since the influence of the change in the posture of the occupant is not biological information, it is preferable to be removed. The trend removal process 82 may, for example, remove the influence of the change in the posture of the occupant.

The data cutting process 83 cuts out the signal obtained by the trend removal process 82 for a predetermined time. For example, the data cutting process 83 cuts out data for a predetermined time as one unit. The signal obtained by the data cutting process 83 is a signal obtained by summing the signals obtained by the trend removal process 82 for a predetermined time.

The first high-pass filter 84, the first low-pass filter 85, the second high-pass filter 86, and the second low-pass filter 87 as frequency filters apply different cutoff frequencies. The first filters and the second filters may be different types of filters.

The cutoff frequency in the frequency filters 84 to 87 is set so that a frequency band including at least heart rate and respiratory rate remains. When the measurement target is only the heart rate, the cutoff frequency may be set so that the frequency band of the heart rate remains, and the frequency band of the respiratory rate may be cut. Further, when the measurement target is only the respiratory rate, the cutoff frequency may be set so that the frequency band of the respiratory rate remains, and the frequency band of the heart rate may be cut. Moreover, the order and the number of the frequency filter may be set as desired.

The noise information may be removed and the biological information may be extracted by the integration process 81, the trend removal process 82, the data cutting process 83, and the frequency filters 84 to 87.

The channel selection process 88 selects a part of the channels with high pressure from the signals obtained by the frequency filters 84 to 87. In this example, the channel selection process 88 selects 16 channels, which are a part of the 32 channels. As described above, the processes of the integration process 81 to the second low-pass filter 87 reduces the noise information and generates a signal in which the biological information is relatively larger than the noise information. Therefore, the channel selection process 88 selects the signals of a part of the 32 channels more influenced by the biological information. The average value, maximum value, and minimum value of the base signals A1 to A32 may be detected, and a part of the channels having high values may be selected.

Next, as shown in FIG. 5, the component analysis part 62 performs a predetermined component analysis based on the multiple pre-processed signals B1 to B16 generated by the pre-processing part 61, and generates multiple component signals C1 to C16.

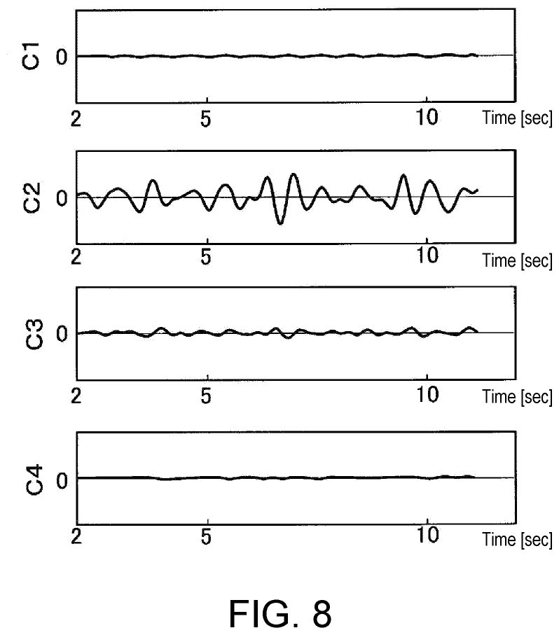

In the predetermined component analysis performed by the component analysis part 62, one of a principal component analysis, an independent component analysis, and a singular value decomposition is performed based on the multiple pre-processed signals B1 to B16, and the multiple component signals C1 to C16 are generated. The principal component analysis is suitable as the predetermined component analysis. FIG. 8 shows a part of the component signals C1 to C4. The component signals C1 to C16 are waveform data for a predetermined time length.

The principle component analysis (PCA) is a type of multivariate analysis, and is a method of searching for components common to multivariate data and creating a synthetic variable (principal component). The independent component analysis (ICA) is an analysis method that expresses data as multiple additive components.

In particular, the principal component analysis may generate the separated component signals C1 to C16 and acquire the component ranks of the component signals C1 to C16. The component rank is higher as the component gives more influence to the input pre-processed signals B1 to B16. In the case of independent component analysis, the component rank may be obtained from the relationship with the base signals A1 to A32.

The component analysis part 62 may separate the component signals into the same number as the number of input signals. That is, in the component analysis part 62, the relationship between the number of components actually included in the pre-processed signals B1 to B16 as input signals and the number of pre-processed signals B1 to B16 as input signals is an important factor. Further, the more the component to be separated is contained in many of the pre-processed signals B1 to B16 which are input signals, the more the component signal to be separated may be acquired.

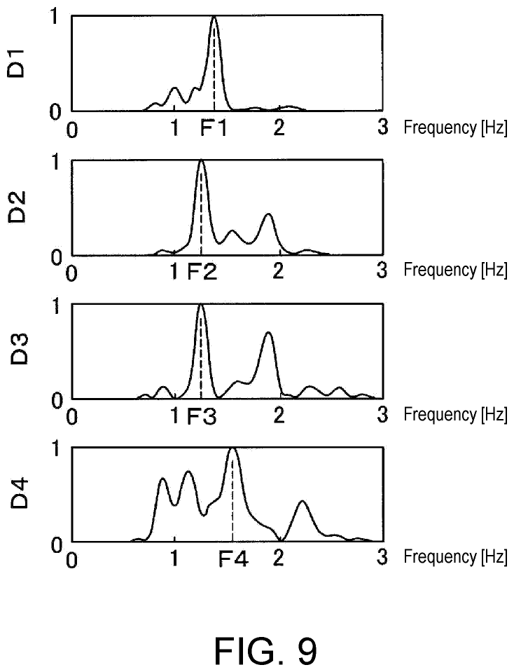

The frequency analysis part 63 will be described with reference to FIGS. 5 and 9. As shown in FIG. 5, the frequency analysis part 63 acquires multiple (16) component signals C1 to C16 as input signals. The frequency analysis part 63 generates multiple power spectra D1 to D16 by performing a FFT process on each of the multiple component signals C1 to C16. Other frequency analysis such as time series modeling, autocorrelation, and wavelet transform may be performed.

The power spectrum D1 is the result of frequency analysis on the component signal C1, and the same applies to the others. A part of the 16 power spectra D1 to D4 are as shown in FIG. 9. The power spectra D1 to D16 represent the signal strength (power) with respect to the frequency. In the power spectra D1 to D16, the maximum signal strength (power) is 1.

Further, the frequency analysis part 63 acquires the respective main frequencies F1 to F16 of the component signals C1 to C16 based on the respective power spectra D1 to D16. The main frequencies F1 to F16 are the primary candidates for the biological information. That is, the frequency analysis part 63 acquires the multiple main frequencies F1 to F16 as the primary candidates for the biological information.

In FIG. 9, the frequencies having the maximum signal strength are the primary candidates F1 to F16. For example, according to FIG. 9, the primary candidate F1 of the component signal C1 is about 1.3 Hz. The main frequencies F1 to F16 are not limited to the frequencies having the maximum signal strength, and may be a spectral band having a predetermined width including the maximum signal strength.

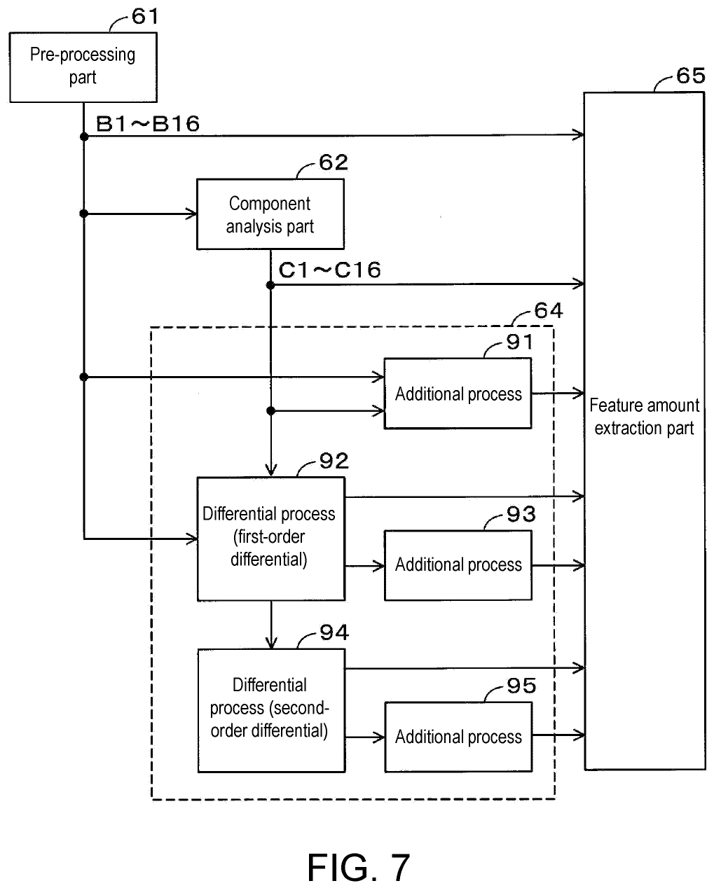

The post-processing part 64 will be described with reference to FIGS. 5 and 7. As shown in FIG. 5, the post-processing part 64 acquires the multiple (16) component signals C1 to C16 as input signals. The post-processing part 64 performs a predetermined post-process on the multiple component signals C1 to C16 as a post-process for a predetermined component analysis by the component analysis part 62, and a large number of post-processed signals Ea1 to Ea16, Eb1 to Eb16, . . . are generated. The predetermined post-process by the post-processing part 64 is a process of generating data used for extracting a feature amount (to be described later).

In this example, the post-processing part 64 further acquires multiple (16) pre-processed signals B1 to B16 as input signals. The post-processing part 64 generates data used for extracting the feature amount for the pre-processed signals B1 to B16. However, the post-processing part 64 does not have to use the pre-processed signals B1 to B16.

In this example, as shown in FIG. 7, the post-processing part 64 performs, as a predetermined post-process, at least one of an additional process 91 for the component signals C1 to C16, a differential process 92 (first-order differential process) for the component signals C1 to C16, an additional process 93 for the first-order differential signals, a differential process 94 (second-order differential process) for the first-order differential signals, and an additional process 95 for the second-order differential signals.

The additional process 91 includes at least one of a frequency analysis process (FFT and the like), time series modeling, a wavelet transform process, an integration process, a correlation process (including autocorrelation and cross-correlation), and a frequency filtering process. When the additional process 91 performs frequency analysis on the multiple component signals C1 to C16, the power spectra D1 to D16 as shown in FIG. 9 are generated as described by the frequency analysis part 63 above. As described above, the power spectra D1 to D16 represent the signal strength (power) with respect to the frequency. In the power spectra D1 to D16, the maximum signal strength (power) is 1.

The differential process 92 performs a differential process on the component signals C1 to C16 to generate first-order differential signals. The additional process 93 performs the same process as the above-mentioned additional process 91 on the first-order differential signals generated by the differential process 92. The differential process 94 performs a differential process on the first-order differential signals to generate second-order differential signals. The additional process 95 performs the same process as the above-mentioned additional process 91 on the second-order differential signals generated by the differential process 94.

Further, the additional process 91, the differential process 92 (first-order differential process), the additional process 93, the differential process 94 (second-order differential process), and the additional process 95 in the post-processing part 64 are performed on the pre-processed signals B1 to B16 in a similar way.

The feature amount extraction part 65 uses the multiple pre-processed signals B1 to B16, the multiple component signals C1 to C16, the multiple post-processed signals D1 to D16, Ea1 to Ea16, Eb1 to Eb16, . . . to extract the features for acquiring the biological information. That is, the feature amount is used as information for extracting the biological information from the multiple primary candidates F1 to F16. In particular, the feature amount extraction part 65 extracts the feature amounts related to the component signals C1 to C16. In particular, in this example, the feature amount extraction part 65 extracts the feature amount related to the primary candidates F1 to F16 generated by the frequency analysis part 63.

For example, the feature amount is used for machine learning for extracting biological information from the multiple primary candidates F1 to F16. That is, the feature quantity is used in the learning process of the determination model that defines the determination condition in the learning phase of machine learning, and is also used in the inference process using the determination model in the inference phase of machine learning. However, when the biological information is acquired by a process different from machine learning, the feature amount is the data used for the process.

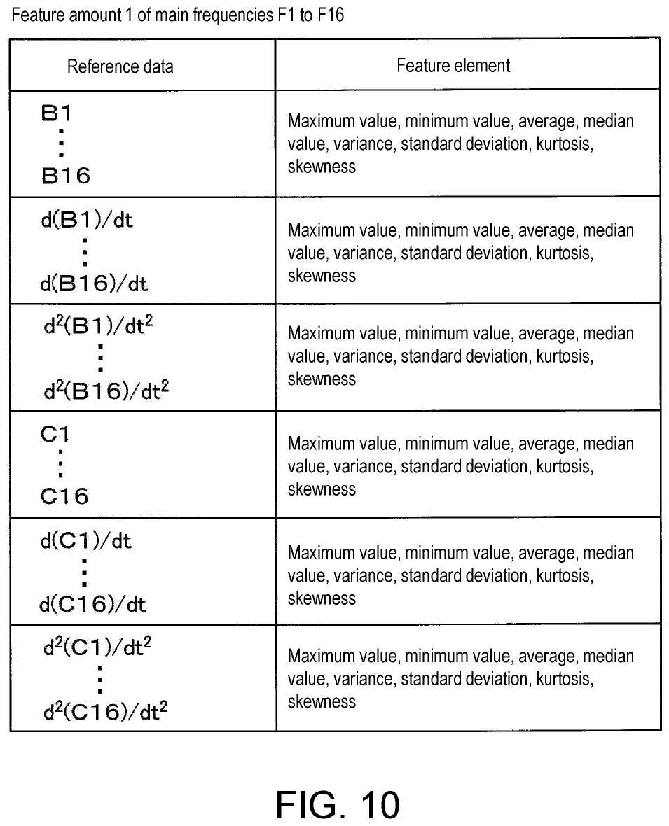

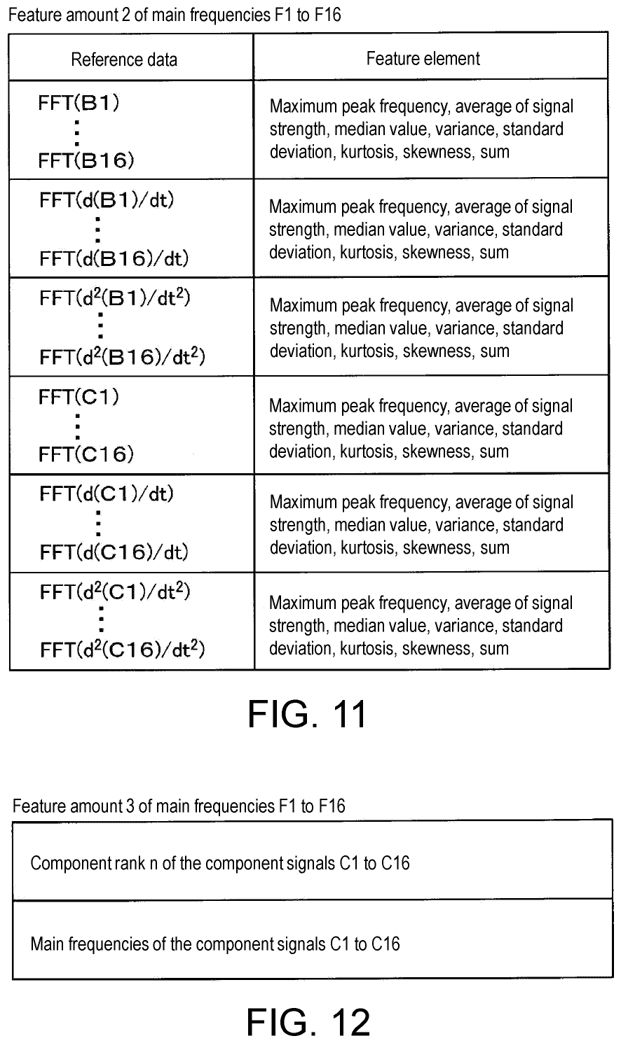

As shown in FIG. 7, the feature amounts include the values obtained from the pre-processed signals B1 to B16, the values obtained from the component signals C1 to C16, and the values obtained from the post-processed signals D1 to D16, Ea1 to Ea16, Eb1 to Eb16 . . . and the like. As shown in FIGS. 10 to 13, there are various candidates for the feature amounts. As the feature amount, one selected from these many candidates may be used. In FIGS. 10 and 11, it is shown that the feature amount is a feature element with respect to the reference data.

For example, the first column of FIG. 10 shows that the pre-processed signals B1 to B16 are used as reference data, and that the maximum value, the minimum value, the average, the median value, the variance, the standard deviation, the kurtosis, the skewness, and the like in the reference data are feature amounts. In this case, the feature amount extraction part 65 inputs the pre-processed signals B1 to B16 generated by the pre-processing part 61, and performs a process on the input signals.

The second column of FIG. 10 shows that the first-order differential signals of the pre-processed signals B1 to B16 are used as reference data, and that the maximum value, the minimum value, the average, the median value, the variance, the standard deviation, the kurtosis, the skewness, and the like in the reference data are feature amounts. In this case, as shown in FIG. 7, the feature amount extraction part 65 inputs the signals generated by the differential process 92 of the post-processing part 64 and performs a process on the input signals to generate the feature amounts.

The third column of FIG. 10 shows that the second-order differential signals of the pre-processed signals B1 to B16 are used as reference data, and that the maximum value, the minimum value, the average, the median value, the variance, the standard deviation, the kurtosis, the skewness, and the like in the reference data are feature amounts. In this case, as shown in FIG. 7, the feature amount extraction part 65 inputs the signals generated by the differential process 94 of the post-processing part 64 and performs a process on the input signals to generate the feature amounts. The m-th order differential (m is 3 or more) of the pre-processed signals B1 to B16 may also be used as reference data.

The fourth to sixth columns of FIG. 10 show that the component signals C1 to C16, the first-order differential signals of the component signals C1 to C16, and the second-order differential signals of the component signals C1 to C16 are used as reference data, and the maximum value, the minimum value, the average, the median value, the variance, the standard deviation, the kurtosis, the skewness, and the like in the reference data are feature amounts. In these cases, as shown in FIG. 7, the feature amount extraction part 65 inputs the signals generated by the component analysis part 62 and the differential processes 92 and 94 of the post-processing part 64, and performs a process on the input signals to generate the feature amounts. The m-th order differential (m is 3 or more) of the component signals C1 to C16 may also be used as reference data. Further, although not shown, the base signals A1 to A32 may be applied as reference data of the feature amounts.

The first column of FIG. 11 shows that the result information FFT (B1) to FFT (B16) obtained by frequency analysis of the pre-processed signals B1 to B16 is used as reference data, and that the maximum peak frequency, the average of the signal strength, the median value, the variance, the standard deviation, the kurtosis, the skewness, and the like in the reference data are feature amounts. In this case, as shown in FIG. 7, the feature amount extraction part 65 inputs the signals generated by the additional process 91 of the post-processing part 64 and performs a process on the input signals to generate the feature amounts.

The second column of FIG. 11 shows that the result information FFT (d(B1)/dt) to FFT (d(B16)/dt) obtained by frequency analysis of the first-order differential signals of the pre-processed signals B1 to B16 is used as reference data, and that the maximum peak frequency, the average of the signal strength, the median value, the variance, the standard deviation, the kurtosis, the skewness, and the like in the reference data are feature amounts. In this case, as shown in FIG. 7, the feature amount extraction part 65 inputs the signals generated by the additional process 93 of the post-processing part 64 and performs a process on the input signals to generate the feature amounts.

The third column of FIG. 11 shows that the result information FFT (d²(B1)/dt²) to FFT (d²(B16)/dt²) obtained by frequency analysis of the second-order differential signals of the pre-processed signals B1 to B16 is used as reference data, and that the maximum peak frequency, the average of the signal strength, the median value, the variance, the standard deviation, the kurtosis, the skewness, and the like in the reference data are feature amounts. In this case, as shown in FIG. 7, the feature amount extraction part 65 inputs the signals generated by the additional process 95 of the post-processing part 64 and performs a process on the input signals to generate the feature amounts. The result information of the frequency analysis for the m-th order differential (m is 3 or more) of the pre-processed signals B1 to B16 may also be used as reference data.

The fourth column of FIG. 11 shows that the result information FFT (C1) to FFT (C16) obtained by frequency analysis of the component signals C1 to C16 is used as reference data, and that the maximum peak frequency, the average of the signal strength, the median value, the variance, the standard deviation, the kurtosis, the skewness, and the like in the reference data are feature amounts. The fifth column of FIG. 11 shows that the result information FFT (d(C1)/dt) to FFT (d(C16)/dt) obtained by frequency analysis of the first-order differential signals of the component signals C1 to C16 are used as reference data, and that the maximum peak frequency, the average of the signal strength, the median value, the variance, the standard deviation, the kurtosis, the skewness, and the like in the reference data are feature amounts.

The sixth column of FIG. 11 shows that the result information FFT (d²(C1)/dt²) to FFT (d²(C16)/dt²) obtained by frequency analysis of the second-order differential signals of the component signals C1 to C16 is used as reference data, and that the maximum peak frequency, the average of the signal strength, the median value, the variance, the standard deviation, the kurtosis, the skewness, and the like in the reference data are feature amounts. The result information of the frequency analysis for the m-th order differential (m is 3 or more) of the component signals C1 to C16 may also be used as reference data.

In the case of the fourth to sixth columns of FIG. 11, as shown in FIG. 7, the feature amount extraction part 65 inputs the signals generated by the additional processes 91, 93 and 95 and performs a process on the input signals to generate the feature amounts.

As shown in FIG. 12, the component rank n of the component signals C1 to C16 and the main frequencies (corresponding to the component frequencies) of the component signals C1 to C16 may be applied as feature amounts. The component rank n is particularly effective when the principal component analysis is performed.

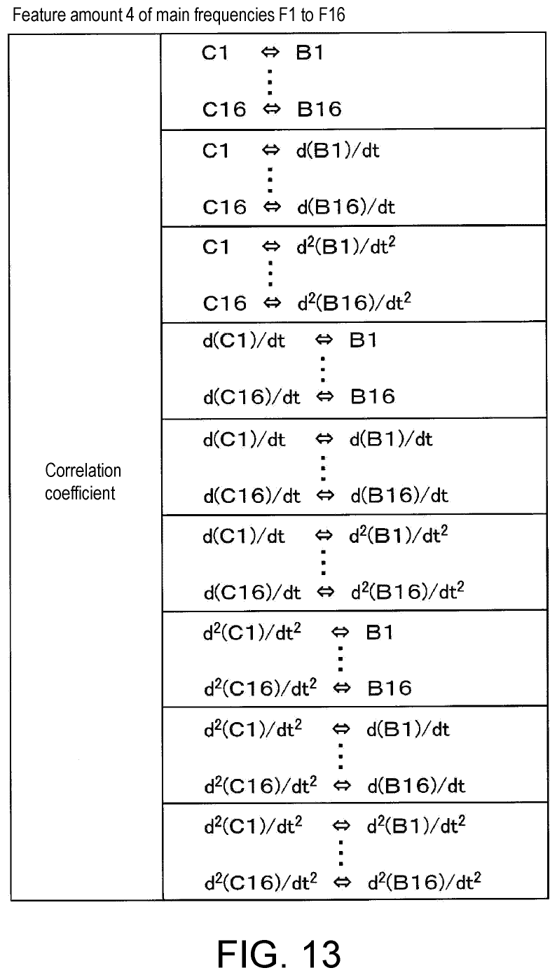

As shown in FIG. 13, the correlation coefficient for two types of signals may be further applied as the feature amount. For example, the first column of FIG. 13 shows that the correlation coefficient between the component signals C1 to C16 and the pre-processed signals B1 to B16 is a feature amount. The second column of FIG. 13 shows that the correlation coefficient between the component signals C1 to C16 and the first-order differential signals of the pre-processed signals B1 to B16 is a feature amount. The third column of FIG. 13 shows that the correlation coefficient between the component signals C1 to C16 and the second-order differential signals of the pre-processed signals B1 to B16 is a feature amount.

Further, the fourth column of FIG. 13 shows that the correlation coefficient between the first-order differential signals of the component signals C1 to C16 and the pre-processed signals B1 to B16 is a feature amount. The fifth column of FIG. 13 shows that the correlation coefficient between the first-order differential signals of the component signals C1 to C16 and the first-order differential signals of the pre-processed signals B1 to B16 is a feature amount. The sixth column of FIG. 13 shows that the correlation coefficient between the first-order differential signals of the component signals C1 to C16 and the second-order differential signals of the pre-processed signals B1 to B16 is a feature amount.

Further, the seventh column of FIG. 13 shows that the correlation coefficient between the second-order differential signals of the component signals C1 to C16 and the pre-processed signals B1 to B16 is a feature amount. The eighth column of FIG. 13 shows that the correlation coefficient between the second-order differential signals of the component signals C1 to C16 and the first-order differential signals of the pre-processed signals B1 to B16 is a feature amount. The ninth column of FIG. 13 shows that the correlation coefficient between the second-order differential signals of the component signals C1 to C16 and the second-order differential signals of the pre-processed signals B1 to B16 is a feature amount.

In the case of each column of FIG. 13, as shown in FIG. 7, the feature amount extraction part 65 inputs the pre-processed signals B1 to B16 generated by the pre-processing part 61, the component signals C1 to C1 generated by the component analysis part 62, and the signals generated by the differential processes 92, 94 and the additional processes 91, 93, 95 of the post-processing part 64, and performs a process on the input signals to generate the feature amounts.

In extracting the above-mentioned feature amounts, the correlation coefficients related to the component signals C1 to C16 and the pre-processed signals B1 to B16 are used. In addition to or instead of the above, the correlation coefficient related to the component signals C1 to C16 and the post-processed signals Ea1 to Ea16, Eb1 to Eb16, . . . and the like may be used as the feature amount.

With reference back to FIG. 5, the configuration of the measuring device 1 will be described. The determination condition storage part 66 of the measuring device 1 stores a determination condition. The determination condition is a condition for determining whether each of the component signals C1 to C16 is biological information. The determination condition is a condition for performing the above determination based on the component signals C1 to C16 and the feature amount.

In particular, in this example, the determination condition is a condition for determining whether each of the primary candidates F1 to F16, which are the main frequencies, is biological information. In this case, the determination condition is, for example, a condition for performing the above determination based on the primary candidates F1 to F16 which are the main frequencies generated by the frequency analysis part 63 and the corresponding feature amount.

In this example, the determination condition storage part 66 stores a determination model that defines the determination condition. The determination model is a model trained by machine learning. For example, the determination model outputs a value indicating whether it is biological information when the primary candidates F1 to F16 and a large number of feature amounts corresponding to the primary candidates F1 to F16 are used as input data. The value indicating whether it is biological information may be a binary value that may distinguish between biological information and non-biological information, or may be a value (determination score) corresponding to the probability of biological information. In this example, the determination model uses a model that may output the determination score. Here, the determination model applies, for example, a random forest or a support vector machine.

The determination model is generated by performing machine learning in advance using the above input data and a teacher label indicating whether the primary candidates F1 to F16 are biological information as a training data set. The teacher label in this case includes at least one of correct answer information which is biological information and incorrect answer information which is not biological information.

The biological information acquisition part 67 acquires frequencies that are biological information by using the multiple primary candidates F1 to F16 generated by the frequency analysis part 63. In this example, the biological information acquisition part 67 applies machine learning to acquire frequencies that are biological information. Specifically, the biological information acquisition part 67 executes an inference phase of machine learning by using a determination model and by using the multiple primary candidates F1 to F16 and feature amounts as input data. Then, the biological information acquisition part 67 determines whether each of the multiple primary candidates F1 to F16 is biological information.

Here, the biological information acquisition part 67 outputs a determination score which is a determination value of whether it is biological information by executing the inference phase of machine learning and determines one piece of biological information using the determination score. However, the biological information acquisition part 67 may perform a correctness determination on whether it is biological information by executing the inference phase of machine learning, and determines the primary candidate determined to be the biological information as the biological information. Further, the biological information acquisition part 67 may determine the primary candidate as biological information according to a predetermined rule without applying machine learning. The detailed process of the biological information acquisition part 67 will be described later.

(4. Process of Biological Information Acquisition Part 67)

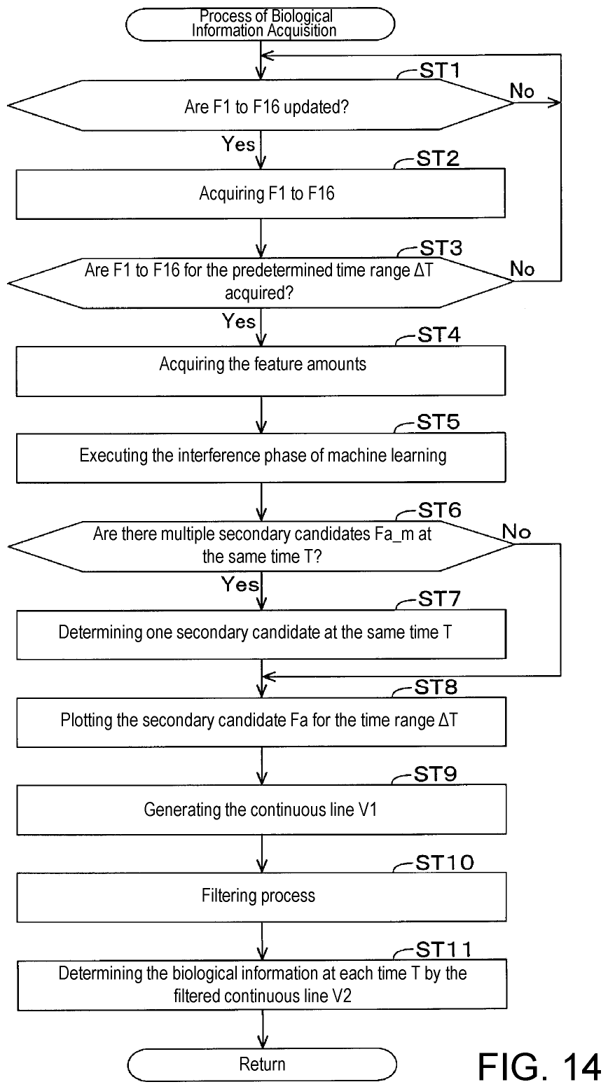

A detailed process of the biological information acquisition part 67 will be described with reference to FIGS. 14 to 21. As shown in FIG. 14, the biological information acquisition part 67 determines whether the primary candidates F1 to F16 have been updated (ST1). If the primary candidates F1 to F16 are not updated (ST1: No), the biological information acquisition part 67 continues the process until the primary candidates F1 to F16 are updated. On the other hand, if the primary candidates F1 to F16 are updated (ST1: Yes), the process proceeds to the next process. That is, the biological information acquisition part 67 proceeds to the next process when the primary candidates F1 to F16 at a new time T are generated.

Subsequently, the biological information acquisition part 67 acquires the primary candidates F1 to F16 at the new time T (ST2). Subsequently, the biological information acquisition part 67 determines whether the primary candidates F1 to F16 for the latest predetermined time range ΔT have been acquired (ST3). If the primary candidates F1 to F16 for the predetermined time range ΔT have not been acquired (ST3: No), the process returns to ST1 again and the process is repeated. That is, the primary candidates F1 to F16 at the new time T are continuously acquired until the primary candidates F1 to F16 for the latest predetermined time range ΔT are acquired.

Subsequently, when the biological information acquisition part 67 acquires the primary candidates F1 to F16 for the predetermined time range ΔT (ST3: Yes), the biological information acquisition part 67 acquires multiple feature amounts extracted by the feature amount extraction part 65 (ST4).

Subsequently, the biological information acquisition part 67 executes the inference phase of machine learning by using the determination model stored in the determination condition storage part 66 and by using the multiple primary candidates F1 to F16 and the multiple feature amounts at each time T as the input data (ST5). Then, the biological information acquisition part 67 outputs a determination value indicating whether each of the multiple primary candidates F1 to F16 at each time T is biological information.

The determination value may be a binary value that may distinguish between biological information and non-biological information, or may be a value (determination score) corresponding to the probability of biological information. The determination score is determined in a range having a predetermined upper and lower limit values. The larger the value of the determination score, that is, the closer to the upper limit value, the higher the probability of biological information.

When a binary value is output as in the former case, the primary candidates F_n (F_n corresponds to F1 to F16) determined to be biological information as a result of executing the inference phase of machine learning is defined as secondary candidates Fa_m. m is a natural number. In this case, the number of the secondary candidates Fa_m is smaller than the number of the primary candidates F1 to F16.

On the other hand, when a determination score is output as in the latter case, all of them may be defined as the secondary candidates Fa_m, or only those whose determination score is larger than the predetermined value may be defined as the secondary candidates Fa_m. Therefore, when all are the secondary candidates Fa_m, the number of the secondary candidates Fa_m is equal to the number of of the primary candidates F_n. On the other hand, when only those whose determination score is larger than a predetermined value are defined as the secondary candidates Fa_m, the number of the secondary candidates Fa_m is smaller than the number of the primary candidates F_n.

When the biological information acquisition part 67 does not apply machine learning, it determines whether each of the multiple primary candidates F1 to F16 at each time T is biological information based on the input data and the determination condition, by a so-called rule-based method.

Subsequently, the biological information acquisition part 67 determines whether there are multiple secondary candidates Fa_m at the same time T (ST6). If there are multiple secondary candidate Fa_m at the same time T (ST6: Yes), one secondary candidate Fa at the same time T is determined by using the multiple secondary candidate Fa_m at the same time T (ST7). On the other hand, when the biological information acquisition part 67 determines that only one secondary candidate Fa_1 at the same time T is biological information (ST6: No), the biological information acquisition part 67 proceeds to the next process (ST8).

Here, the determination of one secondary candidate Fa in step ST7 may be selected from, for example, the following four methods. In a first method for determining one secondary candidate Fa, the biological information acquisition part 67 calculates the arithmetic mean of multiple secondary candidates Fa_m, and determines the arithmetic mean as one secondary candidate Fa. The arithmetic mean Av1 is expressed by equation (1). In equation (1), Xn is a data value, and n is the number of data.

Av1=Σ(Xn)/n . . . (1)

In a second method for determining one secondary candidate Fa, the biological information acquisition part 67 calculates a weighted average in consideration of the determination score, and the weighted average is determined as one secondary candidate Fa. The weighted average Av2 is expressed by equation (2). In equation (2), Xn is a data value, n is the number of data, and Wn is a weight.

Av2=Σ(Wn*Xn)/Wn . . . (2)

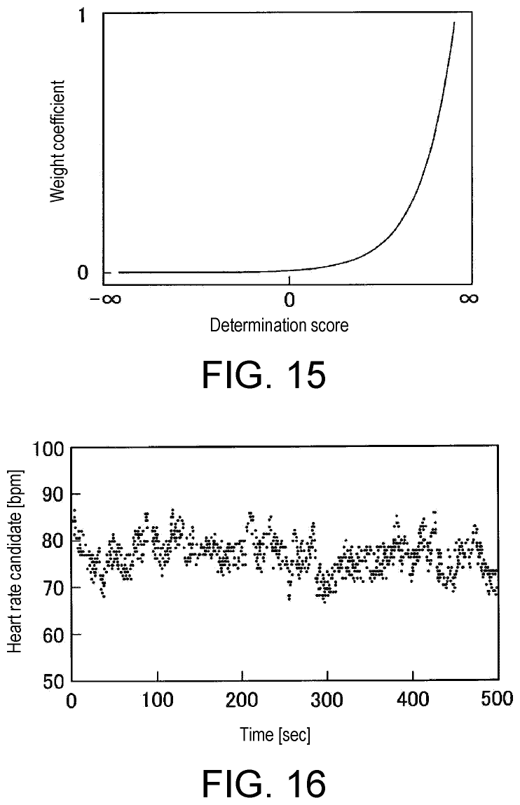

The weight Wn is a value obtained in consideration of the determination score. Specifically, the weight Wn is a value obtained by multiplying the determination score by a softmax function. The softmax function is as shown in FIG. 15. As described above, the larger the value of the determination score, that is, the closer to the upper limit value, the higher the probability of biological information. Therefore, the weight Wn becomes a larger value as the probability of being biological information is higher, and becomes substantially zero when the probability of being biological information is low.

In a third method for determining one secondary candidate Fa, the biological information acquisition part 67 determines a primary candidate F_n with the maximum determination score among the multiple primary candidates F1 to F16 as one secondary candidate Fa.

In a fourth method for determining one secondary candidate Fa, the biological information acquisition part 67 determines one secondary candidate Fa based on the weighted average in consideration of the component rank of the component signals in the principal component analysis or the independent component analysis by the component analysis part 62 for the multiple secondary candidates Fa_m. The weighted average is as shown in the above equation (2). In this case, the weight Wn is a value according to the component rank. For example, the weight Wn is set so that the higher the component rank, the larger the value.

In the above steps ST5 to ST7, the secondary candidate Fa is determined based on multiple primary candidates F_n by applying machine learning. In addition to this, the secondary candidate Fa may be the primary candidate F_n determined to be biological information without applying machine learning. For example, the secondary candidate Fa may select one or more from the multiple primary candidates F_n without relying on machine learning. The secondary candidate Fa may be selected from multiple primary candidates F_n according to a preset rule, or may be randomly selected. The method for selecting the secondary candidate Fa is not limited to the above.

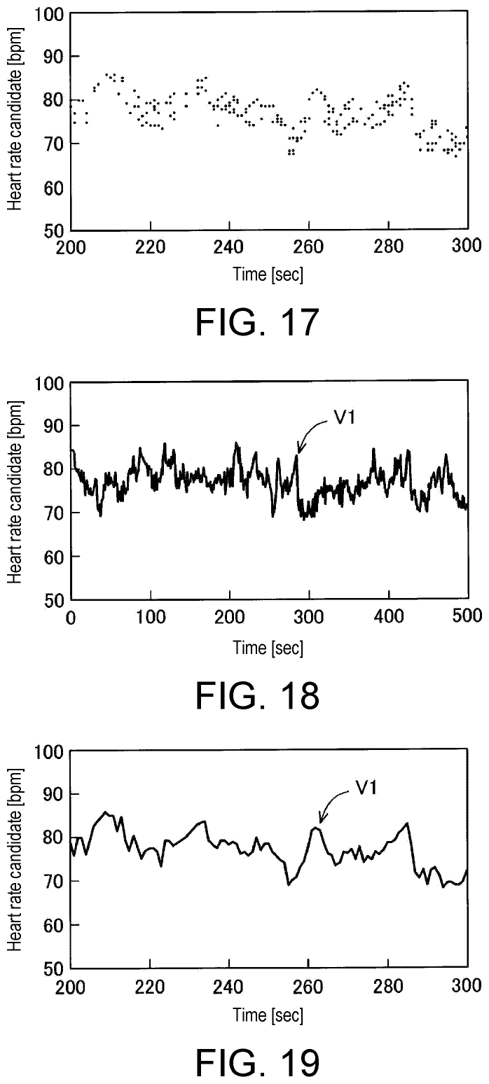

Subsequently, as shown in FIGS. 16 and 17, the biological information acquisition part 67 plots the secondary candidate Fa for the predetermined time range ΔT on a two-dimensional graph (ST8). In the two-dimensional graph, the first axis (horizontal axis) is the time, and the second axis (vertical axis) is the secondary candidate Fa representing biological information. FIGS. 16 and 17 are graphs when the heart rate is targeted as biological information. Here, the human respiratory rate and the heart rate fluctuate with time. Then, in FIGS. 16 and 17, the secondary candidate Fa as the heart rate fluctuates in the range of 70 bpm to 85 bpm with the time.

Here, the biological information acquisition part 67 may perform a data interpolation process, for example, when there is data omission. For example, the biological information acquisition part 67 generates data at a certain time when there is a data omission by using the data before and after the certain time.

Subsequently, the biological information acquisition part 67 generates a continuous line V1 by linearly connecting the secondary candidates Fa at adjacent times in the plotted two-dimensional graph (ST9). The continuous line V1 is as shown in FIGS. 18 and 19.

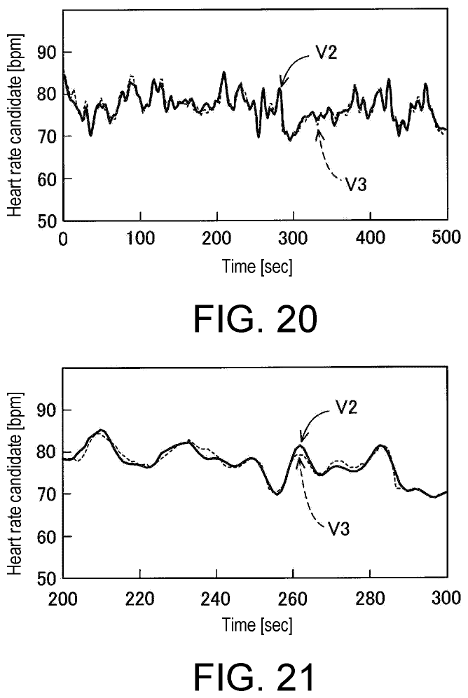

Subsequently, the biological information acquisition part 67 generates a filtered continuous line V2 by subjecting the continuous line V1 to a process by a predetermined frequency filter, for example, a low-pass filter process (ST10). The filtered continuous line V2 is shown by the solid line in FIGS. 20 and 21. Then, the biological information acquisition part 67 determines the biological information at each time T by the filtered continuous line V2 (ST11). That is, the values located on the lines of FIGS. 20 and 21 are the biological information at each time T.

Here, in FIGS. 20 and 21, the actual heart rate is shown by the broken line V3. The actual heart rate is the result of measuring by attaching a heart rate sensor to the occupant. According to FIGS. 20 and 21, it may be seen that the filtered continuous line V2 matches the actual heart rate very well.

Instead of the above embodiment, the biological information acquisition part 67 may perform a process such as FFT, time series modeling, autocorrelation, wavelet transform, and the like on the acquired component signals corresponding to the secondary candidates Fa_m, to calculate the heart rate or the like, which is biological information. Further, when there are multiple secondary candidates Fa_m, the calculated heart rate or the like may be used as the data value Xn for performing the arithmetic mean or the weighted average in step ST7.

(5. Effect)

As described above, it may be seen that the measuring device 1 may acquire biological information with high accuracy. The reason why highly accurate biological information may be acquired will be described. First, the component analysis part 62 of the processing device 60 performs a predetermined component analysis based on the multiple base signals A1 to A32 to generate multiple component signals C1 to C16 configuring the multiple base signals A1 to A32. That is, a part of the generated multiple component signals C1 to C16 becomes signals mainly configured by biological information, and other parts become signals mainly configured by noise information. That is, even if the base signals A1 to A32 include noise information in addition to the biological information, the multiple component signals C1 to C16 are signals in which the biological information and the noise information are separated.

However, it is necessary to determine which of the multiple component signals C1 to C16 is the signal related to biological information. Therefore, the biological information acquisition part 67 of the processing device 60 determines whether the component signals C1 to C16 are biological information. That is, the biological information acquisition part 67 determines which of the multiple component signals C1 to C16 is a signal mainly configured by the biological information by making a determination for each of the multiple component signals C1 to C16. Therefore, the measuring device 1 may measure the biological information with high accuracy.

Further, the pre-processing part 61 of the measuring device 1 performs a process of reducing noise information and a process of selecting a signal in which biological information has a large influence. Using the pre-processed signals B1 to B16 thus obtained, the component analysis part 62 generates the component signals C1 to C16. Therefore, the component analysis part 62 may generate the component signals C1 to C16 in which the biological information and the noise information are separated with high accuracy.

Further, the determination condition stored in the determination condition storage part 66 is used for determining which of the component signals C1 to C16 is the biological information. In particular, the biological information acquisition part 67 uses a determination model, which is a machine learning model that defines the determination condition, to determine whether the main frequencies F1 to F16 of the component signals C1 to C16 are biological information.

The determination model is a model for performing the above determination based on the component signals C1 to C16 and a large number of feature amounts. In particular, the determination model is a model for determining whether the main frequencies F1 to F16 are biological information based on the main frequencies F1 to F16 of the component signals C1 to C16 and the feature amounts. That is, the determination model is a model using the feature amounts related to the main frequencies F1 to F16 in addition to the main frequencies F1 to F16.

Therefore, compared with the case where only the component signals C1 to C16 or the main frequencies F1 to F16 are used, by using the feature amounts in addition to the component signals C1 to C16 or the main frequencies F1 to F16, the biological information may be determined with higher accuracy. That is, by utilizing the multiple component signals C1 to C16 or the main frequencies F1 to F16, the biological information may be acquired with high accuracy.

What is claimed is:

1. A biological information measuring device, comprising:
a plurality of sensors that acquire base signals comprising biological information and noise information; and
a processing device that acquires the biological information based on the base signals,
wherein the processing device comprises:
a component analysis part that performs a predetermined component analysis based on the base signals to generate a plurality of component signals configuring the base signals; and
a biological information acquisition part that determines whether the component signal is the biological information.

2. The biological information measuring device according to claim 1, wherein the processing device further comprises a pre-processing part that performs a predetermined pre-process on the base signals as a pre-process for the predetermined component analysis and generates a plurality of pre-processed signals, and
the component analysis part generates the component signals based on the pre-processed signals.

3. The biological information measuring device according to claim 2, wherein the predetermined pre-process is at least one of an integration process, a trend removal process, a data cutting process, a frequency filter process, a phase difference adjustment process, and a partial signal selection process.

4. The biological information measuring device according to claim 3, wherein the predetermined pre-process comprises a trend removal process and a data cutting process.

5. The biological information measuring device according to claim 1, wherein the component analysis part performs any one of a principal component analysis, an independent component analysis, and a singular value decomposition based on the base signals to generate the component signals.

6. The biological information measuring device according to claim 2, wherein the component analysis part performs any one of a principal component analysis, an independent component analysis, and a singular value decomposition based on the pre-processed signals to generate the component signals.

7. The biological information measuring device according to claim 1, wherein the processing device further comprises a post-processing part that performs a predetermined post-process on the component signals as a post-process of the predetermined component analysis to generate a plurality of post-processed signals.

8. The biological information measuring device according to claim 7, wherein the predetermined post-process is at least one of a differential process, a frequency analysis process, a wavelet transform process, an integration process, a correlation process, and a frequency filter process.

9. The biological information measuring device according to claim 1, wherein the processing device further comprises a feature amount extraction part that extracts a feature amount related to the component signal based on at least one of the base signal and the component signal, and
the biological information acquisition part determines whether the component signal is the biological information based on the component signal and the feature amount.

10. The biological information measuring device according to claim 2, wherein the processing device further comprises a feature amount extraction part that extracts a feature amount related to the component signal based on at least one of the pre-processed signal and the component signal, and
the biological information acquisition part determines whether the component signal is the biological information based on the component signal and the feature amount.

11. The biological information measuring device according to claim 7, wherein the processing device further comprises a feature amount extraction part that extracts a feature amount related to the component signal based on at least one of the base signal, the component signal, and the post-processed component signal, and
the biological information acquisition part determines whether the component signal is the biological information based on the component signal and the feature amount.

12. The biological information measuring device according to claim 2, wherein the processing device further comprises:
a post-processing part that performs a predetermined post-process on the component signals as a post-process of the predetermined component analysis to generate a plurality of post-processed signals; and
a feature amount extraction part that extracts a feature amount related to the component signal based on at least one of the pre-processed signal and the post-processed signal, and
wherein the biological information acquisition part determines whether the component signal is the biological information based on the component signal and the feature amount.

13. The biological information measuring device according to claim 9, wherein the processing device further comprises a determination condition storage part that stores a determination condition for determining whether the component signal is the biological information based on the component signal and the feature amount, and
the biological information acquisition part determines whether the component signal is the biological information based on the component signal, the feature amount, and the determination condition.

14. The biological information measuring device according to claim 13, wherein the processing device further comprises a frequency analysis part that performs a frequency analysis on the component signals to generate a power spectrum, and acquires each main frequency of the component signal as a candidate for the biological information based on the power spectrum, and
the biological information acquisition part selects the biological information from a plurality of the main frequencies.

15. The biological information measuring device according to claim 9, wherein the feature amount is at least one of a maximum value, a minimum value, an average value, a median value, a variance, a standard deviation, a kurtosis, and a skewness in a signal used for feature amount extraction.

16. The biological information measuring device according to claim 9, wherein the feature amount is at least one of a maximum value, a minimum value, an average value, a median value, a variance, a standard deviation, a kurtosis, and a skewness of an n-th-order differential in a signal used for feature amount extraction, where n is a natural number.

17. The biological information measuring device according to claim 9, wherein the feature amount is at least one of a correlation coefficient between the base signal and the component signal, and a correlation coefficient between an n-th-order differential of the base signal and the component signal, and the base signal and an n-th-order differential of the component signal, and an n-th-order differential of the base signal and an n-th-order differential of the component signal, where n is a natural number.

18. The biological information measuring device according to claim 9, wherein the feature amount is a component rank of the component signal in a principal component analysis or an independent component analysis.

19. The biological information measuring device according to claim 9, wherein the feature amount is a component frequency in a principal component analysis or an independent component analysis for each of the component signals.

20. The biological information measuring device according to claim 14, wherein the feature amount is a value obtained based on a signal strength of the main frequency in the power spectrum.

21. The biological information measuring device according to claim 9, wherein the feature amount extraction part performs a frequency analysis for at least one of the base signal, the component signal, an n-th-order differential of the base signal, and n-th-order differential of the component signal, where n is a natural number, to generate a power spectrum, and
the feature amount is at least one of a maximum peak frequency of the power spectrum, and an average, a median value, a variance, a standard deviation, a kurtosis, and a skewness of a signal strength of the power spectrum.

22. The biological information measuring device according to claim 1, wherein when there are a plurality of the component signals determined to be the biological information, the biological information acquisition part determines one piece of the biological information based on an arithmetic mean of a plurality pieces of the biological information determined to be the biological information.

23. The biological information measuring device according to claim 13, wherein the determination condition storage part stores a determination model which defines the determination condition and which outputs a determination score that is a determination value of whether each of the component signals is the biological information, and
the biological information acquisition part determines one piece of the biological information based on a weighted average in consideration of the determination score of each of the component signals.

24. The biological information measuring device according to claim 23, wherein the weighted average is weighted by a value obtained by multiplying the determination score by a softmax function.

25. The biological information measuring device according to claim 13, wherein the determination condition storage part stores a determination model which defines the determination condition and which outputs a determination score that is a determination value of whether each of the component signals is the biological information, and
the biological information acquisition part determines the component signal whose determination score is maximum as one piece of the biological information.

26. The biological information measuring device according to claim 1, wherein when there are a plurality of the component signals determined to be the biological information, the biological information acquisition part determines one piece of the biological information based on a weighted average in consideration of a component rank of the component signal in a principal component analysis or an independent component analysis of the component signals determined to be the biological information.

27. The biological information measuring device according to claim 1, wherein the sensor is any one of a capacitance sensor, a piezoelectric sensor, and a Doppler sensor.
